(19) United States
(12) Patent Application Publication
(10) Pub. No.: US 2016/0346399 A1
(43) Pub. Date: Dec. 1, 2016

(54) DRUG DELIVERY COMPOSITIONS AND METHODS OF USE

(71) Applicant: THE ARIZONA BOARD OF REGENTS ON BEHALF OF THE UNIVERSITY OF ARIZONA, Tucson, AZ (US)

(72) Inventors: Eugene A. Mash, Tucson, AZ (US); Pawel R. Kiela, Tucson, AZ (US); Fayez K. Ghishan, Tucson, AZ (US)

(73) Assignee: THE ARIZONA BOARD OF REGENTS ON BEHALF OF THE UNIVERSITY OF ARIZONA, Tucson, AZ (US)

(21) Appl. No.: 15/233,907

(22) Filed: Aug. 10, 2016

Related U.S. Application Data
(63) Continuation-in-part of application No. 14/271,251, filed on May 6, 2014, now Pat. No. 9,415,110.
(60) Provisional application No. 61/821,166, filed on May 8, 2013.

(51) Int. Cl. A61K 47/48; A61K 9/00; A61K 31/609

(52) U.S. Cl. CPC A61K 47/48092; A61K 47/48061; A61K 31/609; A61K 9/0053

(57) ABSTRACT

The present invention provides a composition comprising a disaccharide where at least one of the hydroxyl groups of the disaccharide is attached to a linker. The terminal portion of the linker also includes a drug that is attached via a functional group that is capable of releasing the drug in vivo. Compositions of the invention are useful for delivering the drug at or near inter alia lower GI tract. In some embodiments, compositions of the invention are used for, but not limited to, treating diseases of the colon, and in particular, inflammatory bowel disease, microscopic colitis, and eosinophilic colitis.

### CROSS-REFERENCE TO RELATED APPLICATIONS

This application is a continuation-in-part application of U.S. patent application Ser. No. 14/271,251, filed May 6, 2014, which claims the priority benefit of U.S. Provisional Application No. 61/821,166, filed May 8, 2013, all of which are incorporated herein by reference in their entirety.

### STATEMENT REGARDING FEDERALLY FUNDED RESEARCH

This invention was made with government support under grant numbers R33 CA095944; P30 CA023074; R01 CA097360; R01 CA123547; and R01 DK067286 awarded by the National Institutes of Health. The government has certain rights in the invention.

### FIELD OF THE INVENTION

This invention relates to compositions comprising a disaccharide where at least one of the hydroxyl groups of the disaccharide is covalently linked to a linker. The terminal portion of the linker also includes a drug that is covalently linked with a functional group that is capable of releasing the drug in vivo. Compositions of the invention are useful for delivering the drug at or near inter alia lower GI tract. In some embodiments, compositions of the invention are used for, but not limited to, treating diseases of the colon, and in particular, inflammatory bowel disease, microscopic colitis, and eosinophilic colitis.

### BACKGROUND OF THE INVENTION

Millions of people in the world suffer from inflammatory bowel disease (IBD).

IBD is a collective term used to describe two gastrointestinal disorders of unknown etiology; Crohn's disease (CD) and ulcerative colitis (UC). Both diseases appear to result from the unrestrained activation of an inflammatory response in the intestine. Ulcerative colitis occurs in the large intestine, while Crohn's disease can involve any segment of the gastrointestinal tract. It has been suggested that the pathogenesis of IBD is multifactorial involving susceptibility genes and environmental factors. Although the causative triggers remain unclear, the role of a persistent and likely dysregulated mucosal immune response is central to the pathogenesis of IBD. It remains unclear whether the persistent inflammation, an intrinsic feature of IBD, reflects a primary aberration in mucosal response or results from an inappropriate persistent stimulation.

The course and prognosis of IBD varies widely. For most patients, it is a chronic condition with symptoms lasting for months to years. IBD is most commonly diagnosed in young adults, but can occur at any age. The clinical symptoms of IBD include intermittent rectal bleeding, fever, abdominal pain, and diarrhea, which may range from mild to severe. Additional common signs of IBD are anemia and weight loss. 10 to 15% of all IBD patients will require surgery over a ten year period. Protracted IBD is a risk factor for colon cancer, and the risk begins to rise significantly after eight to ten years of IBD.

Bowel disorders such as IBD are a significant medical problem, and improved methods of treatment are necessary as no completely satisfactory treatments are currently available.

The first line therapy that often is used for treatment of IBD is 5-aminosalicylic acid (5-ASA). A key to successful treatment is to deliver a high concentration of 5-ASA to the site of inflammation. However, when 5-ASA is administered orally without a carrier or protector, it is nearly completely systemically absorbed in the proximal small intestine prior to reaching the affected area, and is extensively metabolized in intestinal epithelial cells and the liver; it is then excreted in the urine, which may predispose patients to the development of a nephrotic syndrome as a side effect. Therefore, strategies to “protect” orally administered 5-ASA from absorption until it reaches the colon have been developed. These strategies include the use of prodrug, delayed-release formulations (coat the drug with polymers), controlled-release formulations (formulate the 5-ASA as ethylcellulose-coated microgranules), and, more recently, sophisticated formulations that combine both delayed-release and sustained-release mechanisms.

Various strategies have been proposed for targeting orally administered drugs to the colon, including: covalent linkage of a drug with a carrier, including those that enhance stability as well as increasing hydrophilicity; coating with pH-sensitive polymers; formulation of timed released systems; exploitation of carriers that are degraded specifically by colonic bacteria; bioadhesive systems; and osmotic controlled drug delivery systems. Various prodrugs (sulfasalazine, ipsalazine, balsalazide, and olsalazine) have been developed that are aimed to deliver 5-aminosalicylic acid (5-ASA) for localized treatment of inflammatory bowel disease (IBD). Microbially degradable polymers, especially azo-crosslinked polymers, have been investigated for use as coatings for drugs targeted to the colon. Certain plant polysaccharides such as amylose, inulin, pectin, and guar gum remain unaffected in the presence of gastrointestinal enzymes and have been explored as coatings for drugs for the formulation of colon-targeted drug delivery systems. Additionally, combinations of plant polysaccharides with crustacean extract, including chitosan or derivatives thereof, are proving of interest for the development of colonic delivery systems.

The concept of using pH as a trigger to release a drug in the colon is based on the pH conditions that vary continuously down the gastrointestinal (GI) tract. Time-dependent drug delivery systems have been developed that are based on a principle of preventative release of drug until 3-4 hours after leaving the stomach. Redox sensitive polymers and bioadhesive systems have also been exploited to deliver the drugs into the colon.

Other systems for drug delivery to the colon, pH-dependent systems, exploit the generally accepted view that pH of the human GI tract increases progressively from the stomach (pH 1-2 which increases to 4 during digestion), small intestine (pH 6-7) at the site of digestion, and it increases to 7-8 in the distal ileum. The coating of pH-sensitive polymers to the tablets, capsules or pellets provides delayed release and protects the active drug from gastric fluid. The polymers used for colon targeting, however, should be able to withstand the lower pH values of the stomach and of the proximal part of the small intestine and also be able to disintegrate at the neutral or slightly alkaline pH of the terminal ileum and preferably at the ileocecal junction.

Lorenzo-Lamosa et al. (Design of microencapsulated chitosan microspheres for colonic drug delivery. J Control Rel, 52: 109-118, 1998) prepared and demonstrated the efficacy of a system, which combines specific biodegradability and pH dependent release behavior. The system consists of chitosan microcores entrapped within acrylic microspheres containing diclofenac sodium as a model drug. The drug was effectively entrapped within the chitosan microcores using spray drying and then microencapsulated into Eudragit™ L-100 and Eudragit™ S-100 acrylic polymers using an oil-in-oil solvent evaporation method. Release of the drug from chitosan multireservoir system was adjusted by changing the chitosan molecular weight or the type of chitosan salt. Furthermore, by coating the chitosan microcores with Eudragit™, perfect pH-dependent release profiles were attained. Similarly, melt extrusion of a drug with various Eudragit™ polymers in the presence or absence of chitosan, gelling agents or the like has the potential to enable colon-specific release.

Other suitable polymers that are slightly permeable to the active ingredient and water, and exhibit a pH-dependent permeability, include, but are not limited to, EUDRAGIT™ RL, EUDRAGIT™ RS, EUDRAGIT™ L, EUDRAGIT™ S, and EUDRAGIT™ E. See also Dressman, J. B., Amidon, C., Reppas, C. and Shah, V. P., Dissolution testing as a prognostic tool for oral drug absorption: Immediate release dosage forms, Pharm Res, 15: 11-22, 1998.

Polysaccharides, which retain their integrity because they are resistant to the digestive action of gastrointestinal enzymes also have been proposed for encapsulating drugs for colonic drug delivery. The matrices of polysaccharides are assumed to remain intact in the physiological environment of stomach and small intestine but once they reach the colon, they are acted upon by bacterial polysaccharidases. This action results in the degradation of the matrices. This family of natural polymers has an appeal to the area of drug delivery as it is comprised of polymers with a large number of derivatizable groups, a wide range of molecular weights, varying chemical compositions, and for the most part, low toxicity and biodegradability, yet high stability. The most favorable property of these materials is that they are already approved as pharmaceutical excipients. A large number of polysaccharides, such as amylose, guar gum, pectin, chitosan, inulin, cyclodextrins, chondroitin sulfate, dextrans and locust bean gum, as well as modifications thereof, have been investigated for their use in colon-targeted drug delivery systems. The most important factor in the development of polysaccharide derivatives for colon-targeted drug delivery is the selection of a suitable biodegradable polysaccharide. As these polysaccharides are usually soluble in water, they must be made water insoluble by crosslinking or hydrophobic derivatization.

Guar gum is hydrophilic in nature and swells in cold water, forming viscous colloidal dispersions, or sols. This gelling property retards release of the drug from the dosage form and renders it susceptible to degradation in the colonic environment. Homogenized and diluted feces from a human source were incubated with the guar gum to investigate the degradation of the polysaccharide sol by intestinal microflora. It produced a rapid decrease in viscosity and an increase in pH (i.e. became more basic) while no such results were observed when it was incubated with autoclaved fecal homogenates. Guar gum was crosslinked with increasing amounts of trisodium trimetaphosphate to reduce its swelling properties for use as a vehicle in oral delivery formulations. As a result of the crosslinking procedure, guar gum lost its non-ionic nature and became negatively charged. This was demonstrated by methylene blue adsorption studies and swelling studies in sodium chloride solutions with increasing concentrations, in which the hydrogels' network collapsed (Gliko-Kabir, I., Yagen, B., Penhasi, A. and Rubinstein, A., Phosphated crosslinked guar for colon-specific drug delivery. I. Preparation and physicochemical characterization. J Control Rel, 63: 121-127, 2000). Crosslinked guar gum products were analysed to check the efficacy as a colon-specific drug carrier and it was found that the product which was crosslinked with 0.1 molar equivalent of trisodium trimetaphosphate was able to prevent the release of 80% of its hydrocortisone load for at least 6 hours in PBS (pH 6.4). When a mixture of α-galactosidase and β-mannanase was added to the buffer solution, an enhanced release was observed. In vivo degradation studies in the rat caecum showed that despite the chemical modification of guar gum, it retained its enzyme-degrading properties in a crosslinker concentration dependent manner. A novel tablet formulation for oral administration using guar gum as the carrier and indomethacin as a model drug has been investigated for colon targeted drug delivery using in vitro methods. Drug release studies under conditions simulating the gastrointestinal transit have shown that guar gum protects the drug from being released completely in the physiological environment of the stomach and small intestine. Studies in pH 6.8 PBS containing rat caecal contents have demonstrated the susceptibility of guar gum to the colonic bacterial enzyme action with consequent drug release (Rama Prasad, Y. V., Krishnaiah, Y. S. R. and Satyanarayana, S., In vitro evaluation of guar gum as a carrier for colon-specific drug delivery. J Control Rel, 51: 281-287, 1998).

Colon-specific drug delivery also has been proposed using dried amylose films to encapsulate pharmaceutical formulations. Amylose, one of the major fractions of starch, possesses the ability to form films through gelation, when prepared under appropriate conditions. The microstructure of the film is potentially resistant to the action of pancreatic α-amylase but is digested by amylases of the colonic microflora. However, under simulated gastrointestinal conditions, coatings made solely of amylose will become porous and allow drug release. Incorporation of insoluble polymers into the amylose film, to control amylose swelling, provides a solution to this problem. A range of cellulose- and acrylate-based copolymers were assessed, of which a commercially available ethylcellulose (Ethocel) was found to control the swelling most effectively. The in vitro dissolution of various coated pellets under simulated gastric and small intestinal conditions, using commercially available pepsin and pancreatin was determined and demonstrated the resistance of the amylose-Ethocel coat (1:4) to such conditions over a period of 12 h (Milojevic, S., Newton, J. M., Cummings, J. H., Gibson, G. R., Botham, R. L., Ring, S. C., Stockham, M. and Allwood, M. C., Amylose as a coating for drug delivery the colon: Preparation and in vitro evaluation using 5-aminosalicylic acid pellets. J Control Rel, 38: 75-84, 1996).

Chitosan is a high molecular weight polycationic polysaccharide derived from naturally occurring chitin by alkaline deacetylation. Chitosan has favourable biological properties such as nontoxicity, biocompatibility, and biodegradability. Similar to other polysaccharides, it also undergoes degradation by the action of colonic microflora, and hence poses its candidature for colon-targeted drug delivery. Tozaki et al. (Tozaki, H., Odoriba, T., Okada, N., Fujita, T., Terabe, A., Suzuki, T., Okabe, S., Murnishi, S, and Yamamoto, A., Chitosan capsules for colon-specific drug delivery: enhanced localization of 5-aminosalicylic acid in the large intestine accelerates healing of TNBS-induced colitis in rats. J Control Rel, 82, 51-61, 2002) developed colon-specific insulin delivery with chitosan capsules. In vitro drug release experiments from chitosan capsules containing 5(6)-carboxyfluorescein (CF) were carried out by a rotating basket method with slight modifications. The intestinal absorption of insulin was evaluated by measuring the plasma insulin levels and its hypoglycemic effects after oral administration of the chitosan capsules containing insulin and additives. Little release of CF from the capsules was observed in an artificial gastric juice (pH 1), or in an artificial intestinal juice (pH 7). However, the release of CF was markedly increased in the presence of rat caecal contents. This group further evaluated colon-specific insulin delivery using chitosan capsules. It was found that these were stable in the stomach and small intestine but degraded by micro-organisms in rat caecal contents upon entering into the colon, proving their utility as carriers for colon-targeted drug delivery of peptide and non-peptide drugs.

Pectin, a predominantly linear polymer of mainly α-(1→4)-linked D-polygalacturonic acid residues, has been widely investigated as a colon-specific drug delivery entity. It can be broken down by pectinase enzymes produced by anaerobic bacteria of the colon and can control drug release by this principle (Atyabi et al, Carbohyd. Polymers, 2005, 61, 39-51). As pectin is water soluble, efficient colonic delivery requires that the solubility is controlled. Liu et al. (Liu et al, Biomaterials 2003, 24, 3333-3343) demonstrated promising drug delivery potential when pectin was combined with water-insoluble polymers. Previously, Wakerly et al. (Wakerly et al., Pharm. Res., 1996, 13 (8), 1210-1212) identified that a combination of ethylcellulose and pectin could provide protection of a drug in the upper GI tract while allowing enzymatic breakdown and drug release in the colon. Wei et al. (Wei et al., PDA Journal of Pharmaceutical Science and Technology, Vol 61, No. 2, March-April 2007, 121-130) demonstrated that colon-specific controlled release of the water-soluble anticancer agent, 5-fluorouracil, was possible when incorporated into pellets that were coated with various proportions of pectin and ethycellulose (Surlease®).

Redox potential is an expression of the total metabolic and bacterial activity in the colon and it is believed to be insensitive to dietary changes. The mean redox potential in proximal small bowel is −67±90 mV, in the distal small bowel is −196±97 mV and in the colon is −145±72 mV. Thus, microflora-induced changes in the redox potential can be used as a highly selective mechanism for targeting to the colon. Bragger et al. (Investigations into the azo reducing activity of a common colonic microorganism. Int J Pharm, 157: 61-71, 1997) carried out investigations into the azo reducing activity, which could enlighten some factors affecting the bacterial reduction (cleavage) of azo compounds. A common colonic bacterium, Bacteroides fragilis, was used as test organism, and the reductions of azo dyes amaranth, Orange II, tartrazine, and a model azo compound, 4,4′-dihydroxyazobenzene, were studied. It was found that the azo compounds were reduced at different rates, and the rate of reduction could be correlated with the redox potential of the azo compounds. Disulfide compounds can also undergo degradation due to the influence of redox potential in the colon. Noncrosslinked redox-sensitive polymers containing an azo and/or a disulfide linkage in the backbone have been synthesised (Schacht, E. and Wilding, I. R., Process for the preparation of azo- and/or disulfide-containing polymers. Patent: WO 9111175).

The foregoing discussion of prior art derives primarily from U.S. patent publication 2010/0255087 (“the '087 Publication”) to Coulter who proposed oral pharmaceutical compositions comprising mini-capsules containing one or more active pharmaceutical compounds in a liquid, semi-solid or solid core mini-capsule format, wherein the mini-capsules have release profiles intended to release the active pharmaceutical compound at one or more sites along the gastro-intestinal tract where absorption is maximized or therapeutic efficacy is maximized. More particularly, according to the '087 publication, the mini-capsules are formed of or have coatings formed of materials that are sensitive to one or more pH, time, thickness, erosion and bacterial breakdown to achieve a desired release of active pharmaceutical agents along the gastro-intestinal tract.

### SUMMARY OF THE INVENTION

Some aspects of the invention provide a composition comprising a disaccharide, where at least one hydroxyl group of the disaccharide is linked to a linker. In some embodiments, each of the hydroxyl groups of the disaccharide is linked (e.g., covalently bonded) to a linker. The terminal group of the linker is covalently attached or bonded to a drug that is covalently attached via a functional group that is capable of releasing the drug in vivo. By selecting appropriate linkers and/or the functional group, one can design a composition that is suitable for various desired applications. In one particular embodiment of the invention, compositions of the invention are suitable for delivering a drug in the GI tract of a subject. In some instances, the compounds produced are pro-drug forms of active drugs. Exemplary drugs that are useful in compositions of the invention include, but are not limited to, 5-aminosalicylic acid, 5-fluorouracil, imiquimod, regorafenib, prednisolone, butesonide, etc. Release of drug from the pro-drug can be achieved by enzymatic action, such as reduction of azo functional groups by azoreductase enzymes, or reduction of nitro functional groups by nitroreductases, or by conditions of pH or oxygenation state in particular regions of the GI tract. Compositions of the invention are normally suited for oral administration, but can also be administered via other modes known to one skilled in the art.

### DETAILED DESCRIPTION OF THE INVENTION

One particular aspect of the present invention provides a composition comprising a disaccharide where at least one (typically two or more, often three or more, and most often all) of the hydroxyl groups of the disaccharide are linked to a linker. The terminal portion of the linker is attached to a drug via a functional group (that is present on the linker) that is capable of releasing the drug in vivo.

The term disaccharide refers to a carbohydrate composed of two monosaccharides. It is formed when two monosaccharides are covalently linked to form a dimer. The linkage can be a (1→4) bond, a (1→6) bond, a (1→2) bond, etc. between the two monosaccharides. The ring structure (i.e., ring type) of each of the monosaccharide can be independently a pyranose or a furanose. In addition, each of the monosaccharides can be an α- or β-anomer. Exemplary disaccharides that can be used in the present invention include, but are not limited to, sucrose, lactose, maltose, trehalose, cellobiose, lactulose, and chitobiose, etc. Each of the monosaccharides can independently be a ketonic monosaccharide (i.e., ketose), an aldehyde monosaccharide (i.e., aldose), or any type of hexose of the formula C₆H₁₂O₆ or a derivative thereof. Exemplary aldoses that can be used in preparing disaccharides of the invention include, but are not limited to, allose, altrose, glucose, mannose, gulose, idose, galactose, talose, ribose, arabinose, xylose, lyxose, and derivatives thereof. Exemplary ketoses that can be used in preparing disaccharides of the invention include, but are not limited to, psicose, fructose, sorbose, tagatose, ribulose, xylulose, and derivatives thereof. As used herein the term “derivative” refers to a derivative of a monosaccharide in which one or more of the hydroxyl groups is replaced with hydrogen (e.g., 2-deoxy glucose, 5-deoxyglucose, etc.), an amine (e.g., amino sugars) or is replaced with a halogen, such as chloro, fluoro or iodo, (e.g., 5-fluoroglucose, 2-fluoroglucose, 5-chrologlucose, 2-chloroglucose, etc.). Each monosaccharide can also be independently an (L)-isomer or a (D)-isomer.

Some of the specific disaccharides of the invention include compounds of the following formulas:

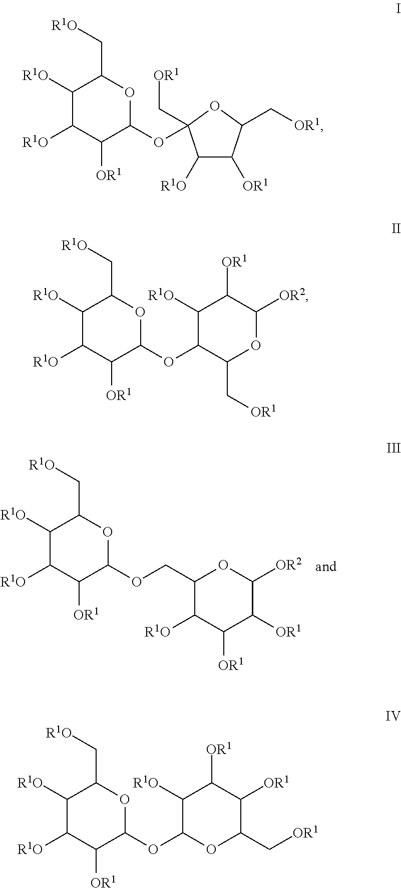

wherein R² is H, a hydroxyl protecting group, or R¹, and each R¹ is independently a linker having chains of atoms selected from the group consisting of C, O, and N, and a drug covalently linked at the terminal portion of said linker with a functional group that is capable of releasing said drug in vivo. Typically, the linker has from about 10 to about 30 atoms in the chain length. The term “about” when referring to a numeric value refers to ±20%, typically ±10%, often ±5% and most often ±1% of the numeric value. It should be noted the functional group is present in the linker itself and is used to link (e.g., bond) the linker to the drug. When the composition is placed in vivo, under appropriate conditions, it undergoes a chemical transformation, thereby cleaving and releasing the drug from the linker and/or the functional group.

As used herein, a hydroxyl protecting group refers to a moiety that, when attached to a hydroxyl group, reduces or prevents reactivity of the hydroxyl group. Examples of hydroxyl protecting groups can be found in T. W. Greene and P. G. M. Wuts, Protective Groups in Organic Synthesis, 3^{rd} edition, John Wiley & Sons, New York, 1999, and Harrison and Harrison et al., Compendium of Synthetic Organic Methods, Vols. 1-8 (John Wiley and Sons, 1971-1996), which are incorporated herein by reference in their entirety. Exemplary hydroxyl protecting groups include, but are not limited to, alkyl (e.g., methyl or ethyl), acyl groups, benzyl and trityl ethers, tetrahydropyranyl ethers, trialkylsilyl ethers and allyl ethers. Representative amino protecting groups include, formyl, acetyl, trifluoroacetyl, benzyl, benzyloxycarbonyl (CBZ), tert-butoxycarbonyl (Boc), trimethyl silyl (TMS), 2-trimethylsilyl-ethanesulfonyl (SES), trityl and substituted trityl groups, allyloxycarbonyl, 9-fluorenylmethyloxycarbonyl (FMOC), nitro-veratryloxycarbonyl (NVOC), and the like.

In some embodiments, the linker comprises at least one cyclic moiety, wherein each of said cyclic moiety is independently selected from the group consisting of aryl, heteroaryl, cycloalkyl, heterocycloalkyl, and a combination thereof, each of which is optionally substituted. The term “aryl” refers to a monovalent monocyclic or bicyclic aromatic hydrocarbon moiety of 6 to 10 ring atoms which is optionally substituted with one or more substituents. When substituted, the aryl group typically has one to three and often one or two substituents, each of which is independently selected. Exemplary substituents include, but are not limited to, alkyl, haloalkyl, heteroalkyl, halo, nitro, cyano, cycloalkyl, -(alkylene)ₙ-COOR (where n is 0 or 1 and R is hydrogen, alkyl or haloalkyl), or -(alkylene)ₙ-CONR^{a}R^{b} (where n is 0 or 1, and R^{a} and R^{b} are, independently of each other, hydrogen or alkyl or R and R′ together with the nitrogen atom to which they are attached form a heterocycloalkyl ring). More specifically the term aryl includes, but is not limited to, phenyl, 1-naphthyl, and 2-naphthyl, each of which is optionally substituted. The term “heteroaryl” means a monovalent monocyclic or bicyclic aromatic moiety of 5 to 12 ring atoms containing one, two, or three ring heteroatoms selected from N, O, or S, the remaining ring atoms being C. The heteroaryl ring is optionally substituted independently with one or more substituents, typically one or two substituents. Exemplary substituents for heteroaryl include substituents for aryl group as discussed above. The terms “heterocyclyl” and “heterocycloalkyl” are used interchangeably herein and mean a saturated cyclic moiety of 3 to 8 ring atoms in which one or two ring atoms are heteroatoms selected from N, O, or S(O)ₙ (where n is an integer from 0 to 2), the remaining ring atoms being C, where one or two C atoms may optionally be replaced by a carbonyl group. The heterocycloalkyl ring may be optionally substituted with one, two, or three substituents, each of which is independently selected. Suitable substituents of heterocycloalkyl include those described above for aryl. The term “cycloalkyl” refers to a saturated monovalent mono- or bicyclic hydrocarbon moiety of 3 to 12, typically 3 to 10 ring carbons. The cycloalkyl may be optionally substituted with one, two, or three substituents, where each substituent is independently selected. Suitable substituents for cycloalkyl include those described above for aryl. The term “alkyl” refers to a saturated linear monovalent hydrocarbon moiety of 1 to 12, preferably 1 to 6, carbon atoms or a saturated branched monovalent hydrocarbon moiety of 3 to 12, preferably 3 to 6, carbon atoms. Exemplary alkyl groups include, but are not limited to, methyl, ethyl, n-propyl, 2-propyl, tert-butyl, pentyl, and the like. The term “alkylene” means a linear saturated divalent hydrocarbon moiety of one to six carbon atoms or a branched saturated divalent hydrocarbon moiety of three to six carbon atoms, e.g., methylene, ethylene, propylene, 2-methylpropylene, pentylene, and the like. The terms “halo” and “halogen” are used interchangeably herein and means fluoro, chloro, bromo, or iodo, typically fluoro or chloro. Similarly, the term “haloalkyl” means alkyl substituted with one or more same or different halo atoms, e.g., —CH₂Cl, —CF₃, —CH₂CF₃, —CH₂CCl₃, and the like, and further includes those alkyl groups such as perfluoroalkyl in which all alkyl hydrogen atoms are replaced by fluorine atoms. The term “heteroalkyl” means a branched or unbranched, cyclic or acyclic saturated alkyl moiety containing carbon, hydrogen and one or more heteroatoms in place of a carbon atom, or optionally one or more heteroatom-containing substituents independently selected from ═O, —OR^{a}, —C(O)R^{a}, NR^{b}R^{c}, (O)NR^{b}R^{c} and —S(O)ₙR^{d} (where n is an integer from 0 to 2). R^{a} is hydrogen, alkyl, haloalkyl, cycloalkyl, cycloalkylalkyl, heterocyclyl, heterocyclylalkyl, aryl, aralkyl, heteroaryl, heteroaralkyl, or acyl. R^{b} is hydrogen, alkyl, haloalkyl, cycloalkyl, cycloalkylalkyl, heterocyclyl, heterocyclylalkyl, aryl, aralkyl, heteroaryl, heteroaralkyl, or acyl. R^{c} is hydrogen, alkyl, haloalkyl, cycloalkyl, cycloalkylalkyl, heterocyclyl, heterocyclylalkyl, aryl, aralkyl, acyl, alkyl sulfonyl, carboxamido, or mono- or di-alkylcarbomoyl. Optionally, R^{b} and R^{c} can be combined together with the nitrogen to which each is attached to form a four-, five-, six- or seven-membered heterocyclic ring (e.g., a pyrrolidinyl, piperidinyl or morpholinyl ring). R^{d} is hydrogen (provided that n is 0), alkyl, haloalkyl, cycloalkyl, cycloalkylalkyl, heterocyclyl, heterocyclylalkyl, aryl, aralkyl, heteroaryl, heteroaralkyl, acyl, amino, monsubstituted amino, disubstituted amino, or hydroxyalkyl. Representative examples of heteroalkyls include, for example, 2-methoxyethyl, benzyloxymethyl, thiophen-2-ylthiomethyl, 2-hydroxyethyl, and 2,3-dihydroxypropyl.

In some embodiments, the linker (including the drug and the functional group) comprises a moiety of the formula:

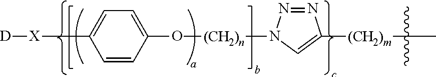

where each of a, b, and c are independently 0 or 1; each of m and n is an integer from 1 to 15, provided that the total number of atoms in said linker is no more than 30; D is a drug; and X is said linkage functional group that is capable of releasing said drug in vivo. In one particular embodiment, a, b, and c are 1, m is 4 and n is 8. Other suitable linkers include polyethylene glycol (e.g., having 2-20, typically 2-15, and often 2-10 ethylene glycol moieties), as well as a mixture of one or more of a straight chain, a branched, and/or a cyclic hydrocarbon optionally having one or more heteroatoms. Thus, the linker can include alkyl chains having one or more cycloalkyl, heterocycloalkyl, heteroaryl, or aryl within the overall chain length. In some embodiments, the linker is a polyethylene glycol linker of the formula (including the functional group and the drug): D-X—O—(CH₂)₂[O—(CH₂)₂]ₙ—, where X and D are those defined herein and n is an integer from 1 to about 20, typically from 1 to about 15 and often from 1 to about 10.

The functional group can be any functional group that can be or is capable of releasing the active drug in vivo. Exemplary functional groups that are useful in compositions of the invention include, but are not limited to, azo (—N═N—), ester (—OC(═O)— or —C(═O)O—), amido (—NHC(═O)— or —C(═O)NH—), sulfonyl or sulfenyl (e.g., —OS(O)ₙ—, where n is 1, 2 or 3), phosphino or phosphatidyl (e.g., —OP(O)ₙ—, where n is 1, 2 or 3), disulfide (—S—S—) etc.

Suitable drugs that can be used in compositions of the invention include any drugs whose functional groups can be used to attach to the linkage functional group and can be released in vivo. Exemplary drugs in compositions of the invention include, but are not limited to, para-aminophenol, 5-aminosalicylic acid, 5-fluorouracil, and imiquimod. In general any drug that has a reactive functional group, such as but not limited to, an amine (e.g., —NR¹R², where each of R¹ and R² is independently H, alkyl, haloalkyl, carboxyl or carbonyl moiety (e.g., —C(═O)R³, where R³ is H, alkyl, haloalkyl, etc.), hydroxyl (—OH), carboxyl (i.e., —CO₂R, where R is H or alkyl), amide (—CONR¹R², where each of R¹ and R² is independently H or alkyl), etc.

Illustrative preparative methods for producing a composition of Formulas I, II, III, and IV are shown in Schemes I, II, III, and IV below, respectively. As can be seen, the chemistry is similar in all four except that for compound of Formulas II and III, the hemiacetal hydroxyl group may be a hydroxyl group protecting group.

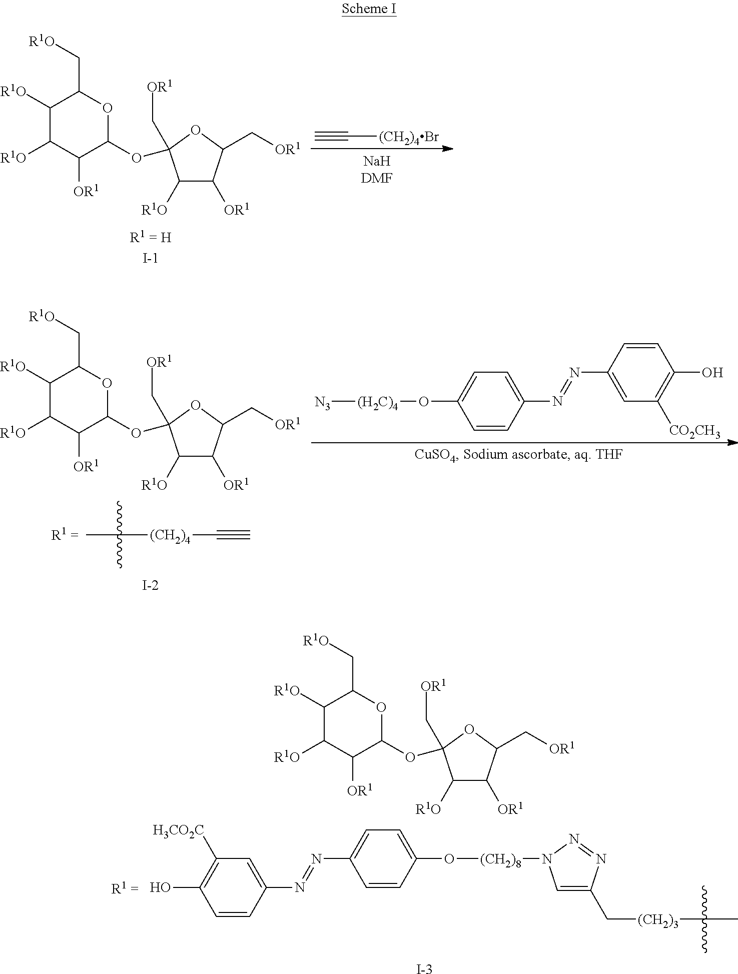

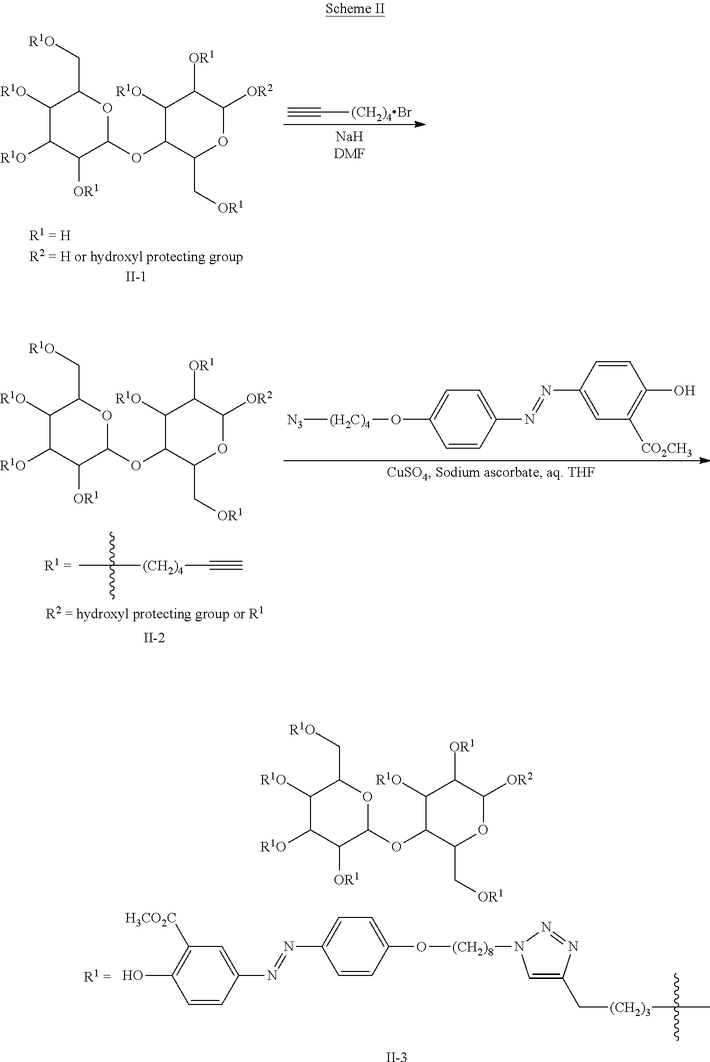

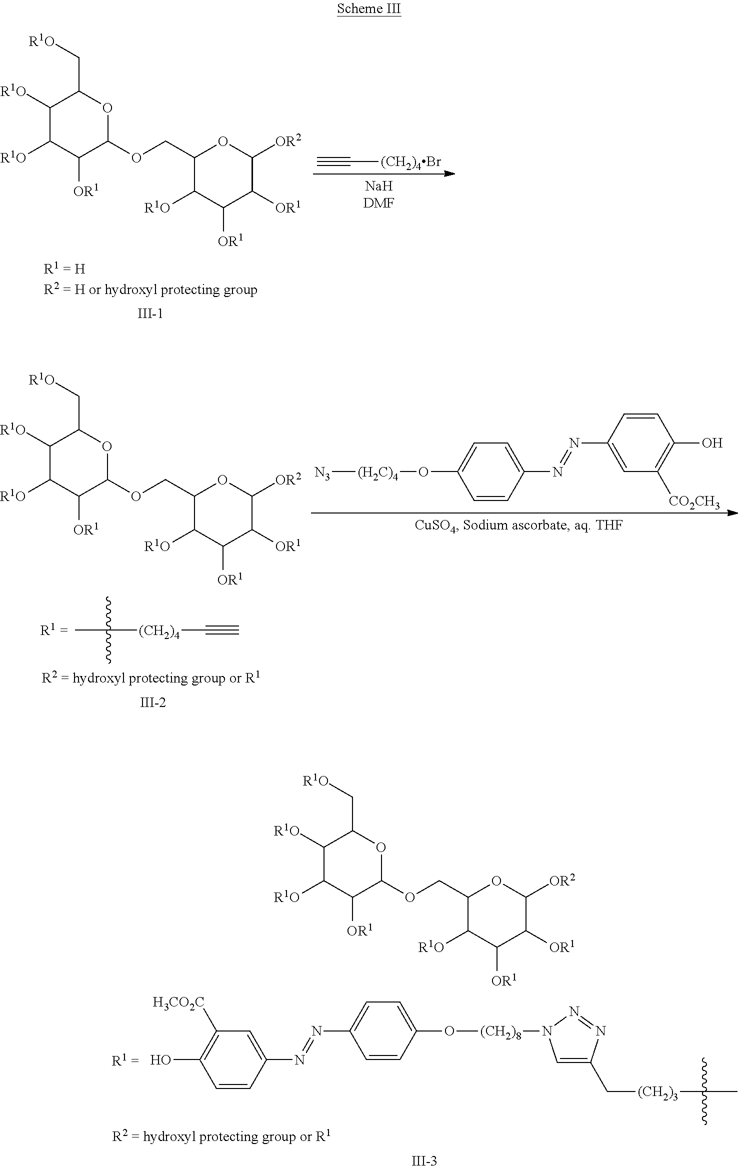

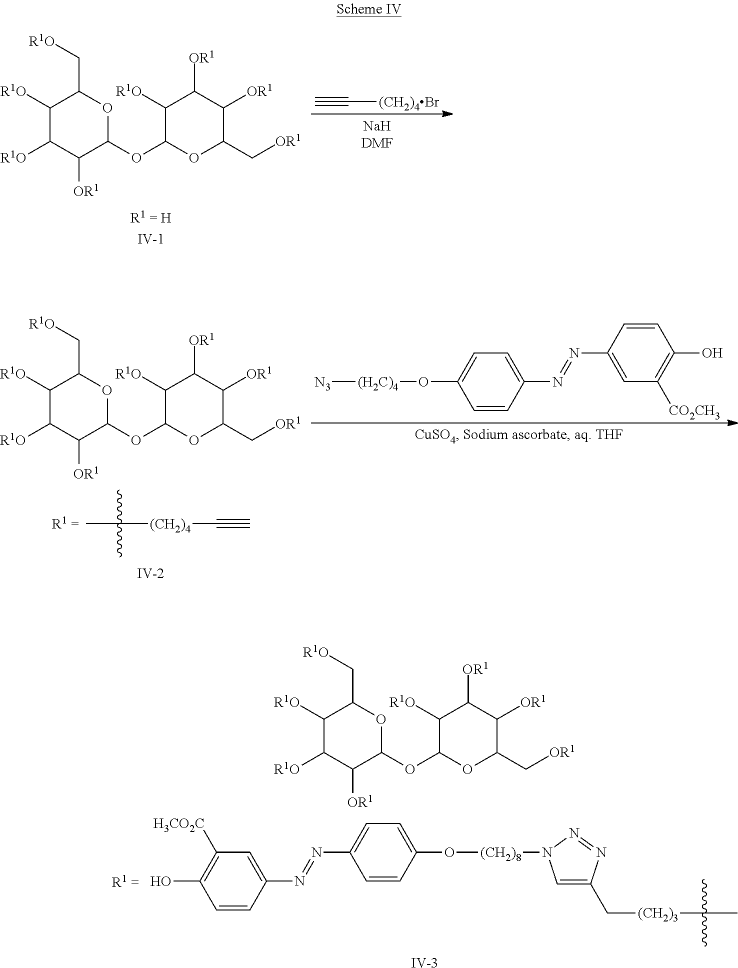

As a brief illustrative example of producing compounds of the invention, a disaccharide of formulas I-1, II-1, III-1, or IV-1 is deprotonated with a strong base, such as sodium hydride in dimethylformamide (DMF). Addition of excess 6-bromohex-1-yne, e.g., 10 equivalents, to the mixture then afforded compound of Formulas I-2, II-2, III-2, or IV-2 respectively. As can be seen, depending on the nature of R² group the disaccharide of formulas II-1 and III-1, R² in compounds of Formulas II-2 and III-2 can be a hydroxyl protecting group or the same R¹ group as in other hydroxyl groups. It should be appreciated that the reaction can be performed in separate steps or a mixture of the disaccharide I-1, II-1, III-1, or IV-1 and a strong base and 6-bromohex-1-yne can be added substantially simultaneously to the reaction vessel.

Suitable strong bases include non-nucleophilic bases in which the corresponding conjugate acid has pKa greater than a hydroxyl group, whose pKa is about 16. Typically, the conjugate acid of the strong base has pKa of about 20 or higher, often pKa of about 25 or higher, and more often pKa of 30 or higher. Exemplary suitable strong bases include, but are not limited to, metal hydrides (e.g., NaH, CaH, KH, LiH, etc.), sterically hindered amide anions (e.g., lithium, sodium, potassium or calcium diisopropylamide), metal alkanes (e.g., tert-butyl lithium, or other butyl metals), etc. Suitable strong bases that can be used in preparing compositions of the invention are well known to one skilled in the art.

Suitable solvents include any non-protic organic solvent such as, but not limited to, tetrahydrofuran (THF), DMF, dimethylsulfoxide (DMSO), diethyl ether, etc., and a combination of two or more such solvents. Generally a polar non-protic solvent is used, such as DMF or DMSO. However, one can use a mixture of a non-polar and a polar non-protic solvents, such as a THF-DMF mixture, a THF-DMSO mixture, etc.

Referring again to Schemes I, II and III, a compound of Formula I-2, II-2 or III-3 is then reacted with an azide group in the presence of Cu(I)-catalyst to promote cyclization reaction to produce the desired triazole-containing compound of Formula I-3, II-3, III-3, or IV-3, respectively. In this manner, a wide variety of compounds of the invention can be produced. As can be seen in Schemes I, II, III, and IV, a Cu(I)-catalyst can be produced in situ by reduction of Cu₂SO₄ with sodium ascorbate. However, it should be appreciated other sources of Cu(I)-catalyst can be used in preparing the triazole-containing compounds of formulas I-3, II-3, III-3, and IV-3.

In schemes I, II, III, and IV, the azo function group (i.e., —N═N—) serves as a functional group that releases the active drug in vivo. As discussed above, other functional groups can also be used to release the active drug in vivo. A specific example of compound of Formulas I, II, III, and IV include, but are not limited to the following specific disaccharide compounds:

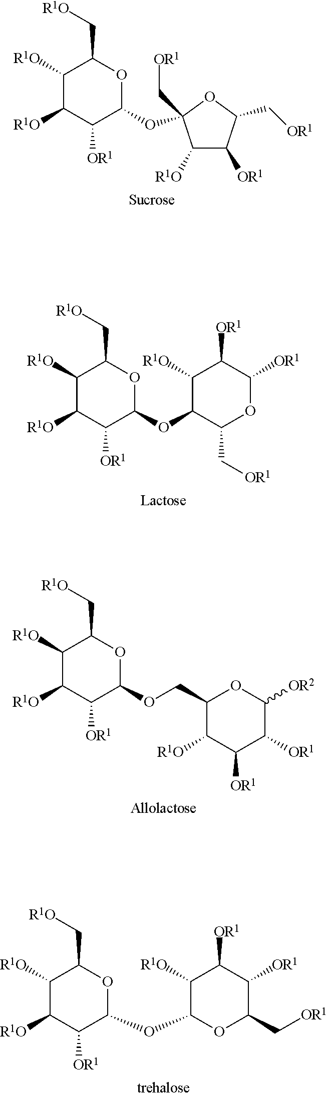

where R¹ and R² are those defined herein.

It should be appreciated, however, the scope of the invention is not limited to these specific disaccharides, but includes any disaccharide compound where at least one hydroxyl group of the disaccharide is covalently linked to a linker, in which a drug is covalently linked at a terminal portion of the linker via a functional group that is capable of releasing said drug in vivo.

Because the primary hydroxyl group has a different reactivity than the secondary hydroxyl group, one can take advantage of this difference in reactivity to selectively protect either the primary hydroxyl group or the secondary hydroxyl groups of compounds of Formulas I-1, II-1, III-1, and IV-1. In this manner, one can readily produce compounds of the invention having the following structures:

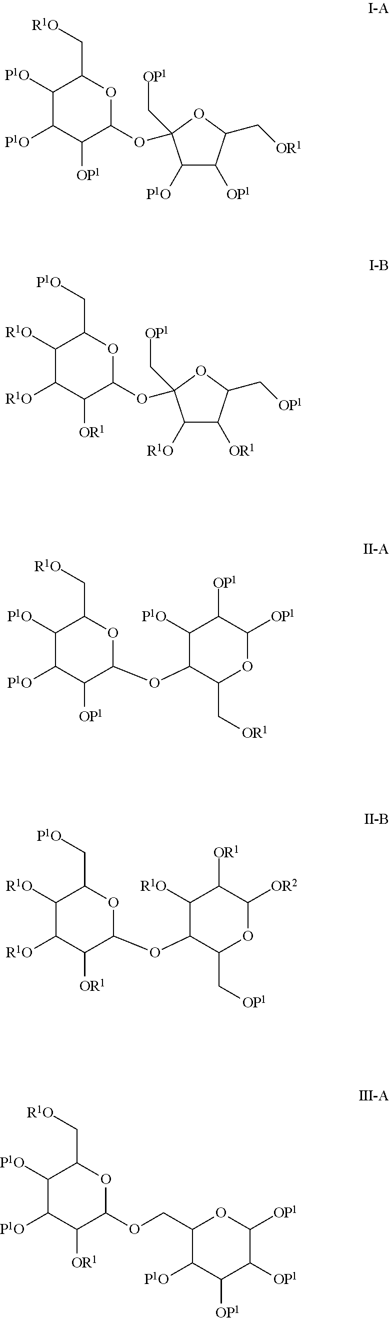

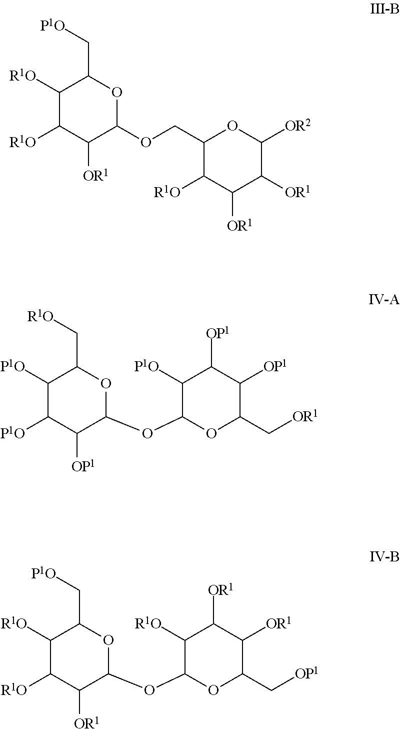

where R¹ is as defined herein and P¹ is a hydroxyl protecting group.

As can be seen above, in compound of Formula III-A only one hydroxyl group of the disaccharide is covalently linked to a linker, in which a drug is covalently linked at a terminal portion of the linker via a functional group that is capable of releasing the drug in vivo. In compound of Formulas I-A, II-A, and IV-A the disaccharide has two hydroxyl groups that are covalently linked to a linker.

Accordingly, compounds of the invention have at least one hydroxyl group of the disaccharide that is covalently linked to a linker, in which a drug is covalently linked at a terminal portion of the linker with a functional group in the linker that is capable of releasing said drug in vivo.

The present invention includes pharmaceutical compositions comprising at least one compound of the invention, or an individual isomer, racemic or non-racemic mixture of isomers or a pharmaceutically acceptable salt or solvate thereof, together with at least one pharmaceutically acceptable carrier, and optionally other therapeutic and/or prophylactic ingredients.

In general, the compounds of the invention are administered in a therapeutically effective amount by any of the accepted modes of administration for agents that serve similar utilities. Suitable dosage ranges are typically 1-500 mg daily, typically 1-100 mg daily, and often 1-30 mg daily, depending on numerous factors such as the severity of the disease to be treated, the age and relative health of the subject, the potency of the compound used, the route and form of administration, the indication towards which the administration is directed, and the preferences and experience of the medical practitioner involved. One of ordinary skill in the art of treating such diseases is typically able, without undue experimentation and in reliance upon personal knowledge and the disclosure of this application, to ascertain a therapeutically effective amount of the compounds of the invention.

Typically, compounds of the invention are administered as pharmaceutical formulations including those suitable for oral (including buccal and sub-lingual), rectal, nasal, topical, pulmonary, vaginal, or parenteral (including intramuscular, intraarterial, intrathecal, subcutaneous and intravenous) administration or in a form suitable for administration by inhalation or insufflation. Typical manner of administration is generally oral using a convenient daily dosage regimen that can be adjusted according to the degree of affliction.

A compound or compounds of the invention, together with one or more conventional adjuvants, carriers, or diluents, can be placed into the form of pharmaceutical compositions and unit dosages. The pharmaceutical compositions and unit dosage forms can be comprised of conventional ingredients in conventional proportions, with or without additional active compounds or principles, and the unit dosage forms can contain any suitable effective amount of the active ingredient commensurate with the intended daily dosage range to be employed. The pharmaceutical compositions can be employed as solids, such as tablets or filled capsules, semisolids, powders, sustained release formulations, or liquids such as solutions, suspensions, emulsions, elixirs, or filled capsules for oral use; or in the form of suppositories for rectal or vaginal administration; or in the form of sterile injectable solutions for parenteral use. Formulations containing about one (1) milligram of active ingredient or, more broadly, about 0.01 to about one hundred (100) milligrams, per tablet, are accordingly suitable representative unit dosage forms.

The compounds of the invention can be formulated in a wide variety of oral administration dosage forms. The pharmaceutical compositions and dosage forms can comprise a compound or compounds of the invention or pharmaceutically acceptable salts thereof as the active component. The pharmaceutically acceptable carriers can be either solid or liquid. Solid form preparations include powders, tablets, pills, capsules, cachets, suppositories, and dispersible granules. A solid carrier can be one or more substances which can also act as diluents, flavoring agents, solubilizers, lubricants, suspending agents, binders, preservatives, tablet disintegrating agents, or an encapsulating material. In powders, the carrier generally is a finely divided solid which is a mixture with the finely divided active component. In tablets, the active component generally is mixed with the carrier having the necessary binding capacity in suitable proportions and compacted in the shape and size desired. The powders and tablets preferably contain from about one (1) to about seventy (70) percent of the active compound. Suitable carriers include but are not limited to magnesium carbonate, magnesium stearate, talc, sugar, lactose, pectin, dextrin, starch, gelatine, tragacanth, methylcellulose, sodium carboxymethylcellulose, a low melting wax, cocoa butter, and the like. The term “preparation” is intended to include the formulation of the active compound with encapsulating material as carrier, providing a capsule in which the active component, with or without carriers, is surrounded by a carrier, which is in association with it. Similarly, cachets and lozenges are included. Tablets, powders, capsules, pills, cachets, and lozenges can be as solid forms suitable for oral administration.

Other forms suitable for oral administration include liquid form preparations including emulsions, syrups, elixirs, aqueous solutions, aqueous suspensions, or solid form preparations which are intended to be converted shortly before use to liquid form preparations. Emulsions can be prepared in solutions, for example, in aqueous propylene glycol solutions or may contain emulsifying agents, for example, such as lecithin, sorbitan monooleate, or acacia. Aqueous solutions can be prepared by dissolving the active component in water and adding suitable colorants, flavors, stabilizers, and thickening agents. Aqueous suspensions can be prepared by dispersing the finely divided active component in water with viscous material, such as natural or synthetic gums, resins, methylcellulose, sodium carboxymethylcellulose, and other well known suspending agents. Solid form preparations include solutions, suspensions, and emulsions, and can contain, in addition to the active component, colorants, flavors, stabilizers, buffers, artificial and natural sweeteners, dispersants, thickeners, solubilizing agents, and the like.

The compounds of the invention can also be formulated for parenteral administration (e.g., by injection, for example bolus injection or continuous infusion) and can be presented in unit dose form in ampoules, pre-filled syringes, small volume infusion or in multi-dose containers with an added preservative. The compositions can take such forms as suspensions, solutions, or emulsions in oily or aqueous vehicles, for example solutions in aqueous polyethylene glycol. Examples of oily or nonaqueous carriers, diluents, solvents or vehicles include propylene glycol, polyethylene glycol, vegetable oils (e.g., olive oil), and injectable organic esters (e.g., ethyl oleate), and can contain formulatory agents such as preserving, wetting, emulsifying or suspending, stabilizing and/or dispersing agents. Alternatively, the active ingredient can be in powder form, obtained by aseptic isolation of sterile solid or by lyophilization from solution for constitution before use with a suitable vehicle, e.g., sterile, pyrogen-free water.

The compounds of the invention can be formulated for topical administration to the epidermis as ointments, creams or lotions, or as a transdermal patch. Ointments and creams can, for example, be formulated with an aqueous or oily base with the addition of suitable thickening and/or gelling agents. Lotions can be formulated with an aqueous or oily base and will in general also contain one or more emulsifying agents, stabilizing agents, dispersing agents, suspending agents, thickening agents, or coloring agents. Formulations suitable for topical administration in the mouth include lozenges comprising active agents in a flavored base, usually sucrose and acacia or tragacanth; pastilles comprising the active ingredient in an inert base such as gelatine and glycerine or sucrose and acacia; and mouthwashes comprising the active ingredient in a suitable liquid carrier.

The compounds of the invention can be formulated for administration as suppositories. A low melting wax, such as a mixture of fatty acid glycerides or cocoa butter is first melted and the active component is dispersed homogeneously, for example, by stirring. The molten homogeneous mixture is then poured into convenient sized molds, allowed to cool, and to solidify.

The compounds of the invention can also be formulated for vaginal administration. Pessaries, tampons, creams, gels, pastes, foams or sprays containing in addition to the active ingredient such carriers as are known in the art to be appropriate.

The compounds of the invention can also be formulated for nasal administration. The solutions or suspensions are applied directly to the nasal cavity by conventional means, for example, with a dropper, pipette or spray. The formulations can be provided in a single or multidose form. In the latter case of a dropper or pipette, this can be achieved by the patient administering an appropriate, predetermined volume of the solution or suspension. In the case of a spray, this can be achieved for example by means of a metering atomizing spray pump.

The compounds of the invention can be formulated for aerosol administration, particularly to the respiratory tract and including intranasal administration. The compound will generally have a small particle size for example of the order of five (5) microns or less. Such a particle size can be obtained by means known in the art, for example by micronization. The active ingredient is provided in a pressurized pack with a suitable propellant such as a chlorofluorocarbon (CFC), for example, dichlorodifluoromethane, trichlorofluoromethane, or dichlorotetrafluoroethane, or carbon dioxide or other suitable gas. The aerosol can conveniently also contain a surfactant such as lecithin. The dose of drug can be controlled by a metered valve. Alternatively the active ingredients can be provided in a form of a dry powder, for example, a powder mix of the compound in a suitable powder base such as lactose, starch, starch derivatives such as hydroxypropylmethyl cellulose and polyvinylpyrrolidine (PVP). The powder carrier typically forms a gel in the nasal cavity. The powder composition can be presented in unit dose form, for example, in capsules or cartridges of e.g., gelatine or blister packs from which the powder can be administered by means of an inhaler.

When desired, formulations can be prepared with enteric coatings adapted for sustained or controlled release administration of the active ingredient. For example, the compounds of the invention can be formulated in transdermal or subcutaneous drug delivery devices. These delivery systems are advantageous when sustained release of the compound is necessary or desired and when patient compliance with a treatment regimen is crucial. Compounds in transdermal delivery systems are frequently attached to a skin-adhesive solid support. The compound of interest can also be combined with a penetration enhancer, e.g., Azone (1-dodecylazacycloheptan-2-one). Sustained release delivery systems can be inserted subcutaneously into the subdermal layer by surgery or injection. The subdermal implants encapsulate the compound in a lipid soluble membrane, e.g., silicone rubber, or a biodegradable polymer, e.g., polylactic acid.

The pharmaceutical preparations are typically in unit dosage forms. In such form, the preparation is often subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

Other suitable pharmaceutical carriers and their formulations are described in Remington: The Science and Practice of Pharmacy 1995, edited by E. W. Martin, Mack Publishing Company, 19th edition, Easton, Pa.

When it is possible that, for use in therapy, therapeutically effective amounts of a compound of the invention, as well as pharmaceutically acceptable salts thereof, can be administered as the raw chemical, it is possible to present the active ingredient as a pharmaceutical composition. Accordingly, the disclosure further provides pharmaceutical compositions, which include therapeutically effective mounts of compounds of the invention or pharmaceutically acceptable salts thereof, and one or more pharmaceutically acceptable carriers, diluents, or excipients. When applied to a combination, the term refers to combined amounts of the active ingredients that result in the therapeutic effect, whether administered in combination, serially, or simultaneously. The compounds of the invention and pharmaceutically acceptable salts thereof, are as described above. The carrier(s), diluent(s), or excipient(s) must be acceptable in the sense of being compatible with the other ingredients of the formulation and not deleterious to the recipient thereof. In accordance with another aspect of the disclosure there is also provided a process for the preparation of a pharmaceutical formulation including admixing a compound of the invention, or a pharmaceutically acceptable salt thereof, with one or more pharmaceutically acceptable carriers, diluents, or excipients.

When the compositions of this disclosure comprise a combination of a compound of the present disclosure and one or more additional therapeutic or prophylactic agent, both the compound and the additional agent are usually present at dosage levels of between about 10 to 150%, and more typically between about 10 and 80% of the dosage normally administered in a monotherapy regimen.

In one particular in vivo experiment, a disaccharide having eight azo-linked precursors to 5-aminosalicylic acid was synthesized and evaluated in a DSS model of colitis in BALB/c mice against sulfasalazine as a control. Two independent experiments verified that the compound of the invention, administered in a dose calculated to result in an equimolar 5-ASA yield, outperformed sulfasalazine in terms of protection from mucosal inflammation and T cell activation. The results indicate compounds of the invention can be used for oral administration of a pro-drug form of 5-aminosalicylic acid. See, also, ACS Medicinal Chemistry Letters, 2012, 3, 710-714.

Additional objects, advantages, and novel features of this invention will become apparent to those skilled in the art upon examination of the following examples thereof, which are not intended to be limiting. In the Examples, procedures that are constructively reduced to practice are described in the present tense, and procedures that have been carried out in the laboratory are set forth in the past tense.

### EXAMPLES

### Example 1

### Preparation of octa-O-(5-hexyn-1-yl)-β-D-fructofuranosyl-α-D-glucopyranoside

To a suspension of NaH (330 mg, 13.7 mmol) in dry dimethylformamide (DMF, 10 mL) under argon were added sucrose (200 mg, 0.58 mmol), 6-bromo-1-hexyne³³ (1.13 g, 6.97 mmol), and tetrabutylammonium bromide (50 mg, 0.15 mmol). The mixture was stirred at room temperature for 48 h. The reaction was quenched with sat NH₄Cl and the mixture extracted with ethyl acetate (3×15 mL). The combined organic extracts were washed with water, brine, dried over Na₂SO₄, and filtered. Removal of volatiles in vacuo afforded a residue that was subjected to silica gel chromatography (63-210 μm) using ethyl acetate/hexanes (2:8) as elutant. This afforded 250 mg (0.25 mmol, 43%) of product as a colorless oil, R_{f} 0.6 (ethyl acetate/hexanes, 3:7), [α]_{D}²⁵ 17.0 (c 0.5, CHCl₃); IR (cm⁻¹) 3308, 2928, 2854, 1213, 1152, 1094; ¹H NMR (500 MHz, CDCl₃) δ 1.57-1.71 (m, 32H), 1.95 (m, 8H), 2.18-2.23 (m, 16H), 3.17 (dd, J=9.5 Hz, J=3.4 Hz, 1H), 3.28 (t, J=9.5 Hz, 1H), 3.35-3.71 (m, 20H), 3.77-3.92 (m, 5H), 4.07 (d, J=7.2 Hz, 1H), 5.50 (d, J=3.8 Hz, 1H); ¹³C NMR (125 MHz, CDCl₃) δ 18.3, 24.7, 25.3, 28.6, 28.7, 29.1, 29.2, 29.6, 31.7, 62.3, 68.4, 68.5, 69.5, 70.2, 70.5, 70.6, 70.8, 71.0, 71.1, 71.7, 72.3, 72.4, 72.7, 79.5, 80.5, 81.6, 82.9, 84.0, 84.2, 84.3, 89.9, 104.3; HRMS (MALDI-TOF) calculated for C₆₀H₈₆NaO_{H} [M+Na]⁺1005.6068, observed 1005.6068.

### Examples 2, 3 and 4

### Lactose, Allolactose and Trehalose Derivatives

Using the procedure of Example 1, octa-O-(5-hexyn-1-yl) derivatives of lactose, allolactose and trehalose are prepared.

### Example 5

### Preparation of 1-(8-bromooctyloxy)-4-nitrobenzene

Anhydrous K₂CO₃ (5.50 g, 39.8 mmol) was added to a stirred solution of 4-nitrophenol (3.69 g, 26.5 mmol) and 1,8-dibromooctane (36.00 g, 132.1 mmol) in acetone. The reaction mixture was heated at reflux for 12 h, then cooled to ambient temperature and filtered. The residue was washed with acetone (3×15 mL) and the combined organic layers evaporated in vacuo. The residue was dissolved in EtOAc (50 mL), the solution washed with water (2×20 mL), brine, dried over Na₂SO₄, filtered, volatiles evaporated in vacuo, and the residue subjected to column chromatography on silica gel (230-400 mesh) using hexanes as elutant to afford 7.75 g (23.5 mmol, 89%) of product as a low melting solid, mp 39-40° C. ¹H NMR (500 MHz, CDCl₃) δ 1.35-1.50 (m, 8H), 1.79-1.89 (m, 4H), 3.41 (t, J=6.5 Hz, 2H), 4.04 (t, J=6.5 Hz, 2H), 6.93 (d, J=10 Hz, 2H), 8.18 (d, J=9.5, 2H); ¹³C NMR (125 MHz, CDCl₃) δ 27.9, 28.5, 28.8, 29.0, 29.2, 32.6, 33.9, 68.7, 114.3, 125.8, 129.3, 141.2, 164.1; HRMS (EI) calculated for C₁₄H₂₀BrNO₃ [M]⁺. 329.6027, observed 329.6040.

### Example 6

### Preparation of 4-(8-bromooctyloxy)aniline

To a glass hydrogenation vessel were added 1-(8-bromooctyloxy)-4-nitrobenzene (5.00 g, 15.1 mmol) and absolute ethanol (50 mL). Carefully, 10% Pd/C (0.5 g) was added, the vessel was charged with hydrogen (40 psi), and the mixture was shaken on a Parr hydrogenation apparatus. After 3 h the mixture was diluted with CH₂Cl₂ (25 mL), filtered through a Celite pad, and volatiles removed in vacuo to give 4.20 g (14.0 mmol, 93%), of 6 as a pale pink solid, mp 61-63° C. This material was used without purification in the next reaction. ¹H NMR (500 MHz, CDCl₃) δ 1.27-1.44 (m, 8H), 1.75-1.84 (m, 4H), 3.39 (t, J=5.0 Hz, 2H), 3.89 (t, J=6.5 Hz, 2H), 6.77-6.88 (m, 2H); ¹³C NMR (125 MHz, CDCl₃) δ 22.7, 25.9, 26.0, 28.1, 28.7, 29.2, 29.4, 31.8, 32.8, 34.0, 68.5, 115.7, 118.6, 135.2, 154.1; HRMS (ESI) calculated for C₁₄H₂₃BrNO [M+H]⁺ 300.0957, observed 300.0963.

### Example 7

### Preparation of 5-((4-(8-bromooctyloxy)phenyl)diazenyl)-2-hydroxybenzoic acid

4-(8-Bromooctyloxy)aniline (2.30 g, 7.7 mmol) was suspended in a mixture of concentrated hydrochloric acid (8 mL) and water (75 mL). The resulting solution was cooled to 0° C. in an ice bath. Sodium nitrite (0.83 g, 12.1 mmol) in water (10 mL) was added dropwise to the reaction mixture with rapid stirring over about 20 min. The reaction mixture was stirred for an additional 20 min while salicylic acid (3.68 g, 26.6 mmol) was dissolved in an aqueous NaOH solution (8.0 g NaOH in 100 mL H₂O). This basic salicylic acid solution was vigorously stirred at 0° C. and the solution of the diazonium salt added dropwise. The pH was maintained at 12-14 by adding 8M NaOH solution. After the addition was complete, the solution was allowed to warm to room temperature and was stirred for an additional 30 min. The mixture was diluted with EtOAc (100 mL), washed with water (3×20 mL), 5% NaHCO₃ (2×20 mL), brine, dried over Na₂SO₄, and filtered. Volatiles were removed in vacuo and the residue subjected to column chromatography on silica gel 60 (230-400 mesh) using EtOAc and methanol (98:2) as elutant. The product (1.40 g, 3.1 mmol, 40%) was obtained as a red-brown solid, mp 180-182° C. ¹H NMR (500 MHz, CDCl₃) δ 1.25-1.44 (m, 8H), 1.72-1.85 (m, 4H), 3.38 (t, J=7 Hz, 2H), 3.93 (t, J=6.5 Hz, 2H), 6.86-6.91 (m, 3H), 7.69 (d, J=9 Hz, 2H), 7.84 (dd, J=10 Hz, 2 Hz, 1H), 8.42 (s, 1H); ¹³C NMR (125 MHz, CDCl₃) δ 25.8, 25.9, 28.0, 28.5, 29.0, 29.1, 29.2, 31.7, 32.7, 33.9, 35.2, 68.1, 113.3, 118.4, 123.1, 124.0, 126.1, 127.5, 144.9, 146.5, 161.0; HRMS (ESI) calculated for C₂₁H₂₆BrN₂O₄ (M+H)⁺ 449.1070, observed 449.1063.

### Example 8

### Preparation of 5-((4-(8-Azidooctyloxy)phenyl)diazenyl)-2-hydroxybenzoic acid

5-((4-(8-Bromooctyloxy)phenyl)diazenyl)-2-hydroxybenzoic acid (2.50 g, 5.6 mmol) was dissolved in DMSO (40 mL) and NaN₃ (1.10 g, 16.9 mmol) was added slowly to the reaction mixture with stirring at room temperature. After 48 h, the mixture was diluted with EtOAc (100 mL), washed with water (4×20 mL), brine (20 mL), dried over Na₂SO₄, and filtered. Volatiles were removed in vacuo and the residue subjected to column chromatography on silica gel 60 (230-400 mesh) using EtOAc and methanol (98:2) as the elutant to give 2.10 g (5.1 mmol, 91%) of the product as a red-brown solid, mp 54-56° C. ¹H NMR (500 MHz, CDCl₃) δ 1.27-1.58 (m, 8H), 1.62-1.64 (m, 2H), 1.81-1.86 (m, 2H), 3.31 (t, J=5 Hz, 2H), 4.06 (t, J=6.5 Hz, 2H), 6.88-7.01 (m, 3H), 7.84 (d, J=10 Hz, 2H), 7.91 (dd, J=15 Hz, 2.5 Hz, 1H), 8.47 (d, J=2.5 Hz, 1H); ¹³C NMR (125 MHz, CDCl₃+CD₃OD) δ 22.0, 25.3, 25.4, 28.5, 28.6, 28.8, 31.2, 50.8, 67.7, 114.1, 116.5, 117.5, 123.6, 126.2, 144.6, 146.2, 160.7, 175.0; HRMS (ESI) calculated for C₂ᵢH₂₆N₅O₄ (M+H)⁺ 412.1979, observed 412.1971.

### Example 9

### Methyl 5-((4-(8-Azidooctyloxy)phenyl)diazenyl-2-hydroxybenzoate

To a stirred solution of 5-((4-(8-azidooctyloxy)phenyl)diazenyl)-2-hydroxybenzoic acid (400 mg, 0.97 mmol) in acetone (20 mL) was added powdered anhydrous potassium carbonate (270 mg, 1.94 mmol). Dimethyl sulfate (92 mg, 0.97 mmol) was then added dropwise at room temperature. The reaction mixture was heated at reflux for 20 min and then allowed to cool to ambient temperature. Volatiles were removed in vacuo, the residue taken up in EtOAc (40 mL), the solution washed with water (2×20 mL), brine, dried over Na₂SO₄, and filtered. Volatiles were removed in vacuo and the residue subjected to column chromatography on silica gel 60 (230-400 mesh) using EtOAc and hexanes (95:5) as the elutant to give 210 mg (0.49 mmol, 50%) of the product as a yellow solid, mp 58-60° C. ¹H NMR (500 MHz, CDCl₃) δ 1.39-1.51 (m, 8H), 1.64 (qt, 2H), 1.83 (qt, 2H), 3.28 (t, J=7 Hz, 2H), 4.02 (s, 3H), 4.05 (t, J=6.5 Hz, 2H), 7.88 (d, J=9 Hz, 2H), 8.07 (dd, J=9 Hz, 2.5 Hz, 1H), 8.42 (d, J=2.5 Hz, 1H), 11.06 (s, 1H); ¹³C NMR (125 MHz, CDCl₃) δ 25.9, 26.7, 28.8, 29.1, 29.2, 51.5, 52.5, 68.2, 112.4, 114.7, 118.3, 124.5, 126.0, 128.7, 145.5, 146.7, 161.5, 163.3, 170.3; HRMS (ESI) calculated for C₂₂H₂₈N₅O₄ (M+H)⁺ 426.2135, observed 426.2141.

### Example 10

### Preparation of Octatriazole-Octaester

A mixture of THF and water (9:1, 20 mL) was degassed with argon for 10 min. Methyl 5-((4-(8-Azidooctyloxy)phenyl)diazenyl)-2-hydroxybenzoate (880 mg, 2.07 mmol) and octa-O-(5-hexyn-1-yl)-β-D-fructofuranosyl-α-D-glucopyranoside (200 mg, 0.203 mmol) were added and degassing was continued for 5 min. Copper sulfate (26 mg, 0.02 mmol, 10 mol %) and sodium ascorbate (64 mg, 0.04 mmol, 20 mol %) were added and the solution was stirred under argon. Progress of the reaction was monitored by TLC using 10% methanol in CH₂Cl₂ plus 0.1% ammonium hydroxide. After 12 h the mixture was diluted with EtOAc (50 mL), washed with water (3×50 mL), brine, dried over anhydrous Na₂SO₄, filtered, and volatiles removed in vacuo. The residue was subjected to column chromatography on silica gel 60 (230-400 mesh) eluted with 20% EtOAc and hexanes (250 mL) and with 10% methanol in CH₂Cl₂ plus 0.1% ammonium hydroxide to give 600 mg (0.137 mmol, 68%) of the product as a red-brown solid, mp 50-51° C. ¹H NMR (500 MHz, CDCl₃) δ 1.20-1.75 (m, 144H), 2.56-2.84 (m, 16H), 3.10-4.10 (m, 13H), 3.75-4.15 (m, 40H), 4.25-4.30 (m, 16H), 5.49 (m, 1H), 6.90-7.10 (m, 24H), 7.30-7.40 (m, 8H), 7.90-8.10 (m, 24H), 8.35-8.40 (m, 8H), 11.00-11.10 (m, 8H); ¹³C NMR (125 MHz, CDCl₃) δ 25.29, 25.40, 25.43, 25.46, 25.58, 25.61, 25.65, 25.66, 25.71, 25.82, 25.86, 26.08, 26.17, 26.20, 26.23, 26.26, 26.29, 26.35, 26.41, 28.88, 29.07, 29.59, 29.61, 29.69, 29.76, 30.28, 30.85, 32.12, 50.03, 52.47, 68.13, 70.52, 70.57, 71.03, 71.31, 72.51, 72.63, 72.95, 76.72, 76.98, 77.23, 112.26, 114.57, 118.23, 120.60, 124.42, 125.89, 128.59, 145.34, 146.54, 161.34, 163.19, 170.36; HRMS (MALDI TOF) calculated for C₂₃₆H₃₀₂N₄₀NaO₄₃ 4410.1 (M+Na)⁺, observed 4409.5 (average mass).

### Examples 11, 12, and 13

### Preparation of Octatriazole-Octaester Derivatives of Lactose, Allolactose, and Trehalose

Using the procedure of Example 10, but replacing sucrose derivative of Example 10 with lactose, allolactose, and trehalose derivatives prepared in Examples 2, 3, and 4 above, octatriazole-octaesters of lactose, allolactose, and trehalose are prepared.

### Example 14

### Acid Stability of Octatriazole-Octaester

Octatriazole-octaester (1 mg/mL) was dissolved in in 1:9 THF-aqueous 2N acetic acid (pH approximately 2.2) and the solution kept at room temperature under air. Aliquots were removed at 1 h, 6 h, 18 h, and 24 h, extracted with ethyl acetate (which removed all the color from the aqueous phase), and the extracts analyzed by TLC and finally by MALDI-TOF mass spectrometry. No decomposition was observed.

### Example 15

### Preparation of Octatriazole-Octaacid

Octatriazole-octaester (600 mg, 0.137 mmol) was dissolved in a mixture of THF and methanol (9:1, 75 mL), aqueous 2N LiOH (25 mL) was added, and the reaction mixture was stirred at room temperature. Progress of the reaction was monitored by TLC using 10% methanol in CH₂Cl₂ plus 0.1% ammonium hydroxide. After 24 h, the mixture was extracted with EtOAc (2×75 mL), the organic phases combined, washed with water (50 mL), brine, dried over anhydrous Na₂SO₄, filtered, and volatiles removed in vacuo to leave the product as a yellow viscous liquid (538 mg, 0.12 mmol, 90%) which was used in the next step without purification. ¹H NMR (500 MHz, DMSO) δ 1.10-1.80 (m, 144H), 2.50-2.60 (m, 16H), 3.00-4.40 (m, 45H), 5.36 (m, 1H), 6.90-7.20 (m, 24H), 7.65-8.40 (m, 40H); ¹³C NMR (125 MHz, DMSO) δ 24.74, 24.81, 24.88, 25.31, 25.53, 25.58, 25.63, 25.77, 28.29, 28.51, 28.73, 28.99, 29.16, 29.21, 29.26, 29.67, 29.87, 30.25, 31.98, 33.97, 34.20, 34.70, 39.00, 39.17, 39.33, 39.50, 39.67, 39.76, 39.83, 39.92, 40.00, 43.05, 49.08, 54.88, 60.39, 65.89, 67.82, 113.75, 114.77, 114.93, 118.07, 121.33, 122.36, 124.18, 124.24, 125.15, 125.83140.69, 128.41, 139.06, 140.61, 142.68, 144.38, 145.62, 145.84, 146.60, 148.06, 152.64, 161.02, 163.20, 171.44, 186.04; HRMS (MALDI TOF) calculated for C₂₂₈H₂₈₆N₄₀NaO₄₃ (M+Na)⁺4298.0, observed 4299.3 (average mass).

### Example 16

### Preparation of Octatriazole Sodium Salt

Octatriazole-octaacid from the previous section was dissolved in a mixture of acetonitrile and water (1:9) plus a few drops of DMSO and the solution loaded onto a column packed with 150 g of Dowex 50w-8x (sodium form). The column was eluted with distilled water (300 mL), the volume of the eluent reduced in vacuo to approximately 50 mL, and the solution subjected to lypholyzation to give 480 mg (0.10 mmol, 90%) of the product as a non-hygroscopic bright yellow solid, mp 74-76° C. ¹H NMR (500 MHz, DMSO) δ 1.10-1.80 (m, 128H), 2.40-2.60 (m, 32H), 3.00-4.30 (m, 45H), 5.30 (m, 1H), 6.90-7.40 (m, 24H), 7.60-7.80 (m, 32H), 8.20-8.30 (m, 8H); HRMS (MALDI TOF) calculated for C₂₂₈H₂₈₆N₄₀NaO₄₃ (M+Na)⁺4298.0, observed 4298.2 (average mass).

### Example 17

### In Vivo Mouse Model Study

In the first study, Dextran Sulfate Sodium Salt (DSS) was added to the drinking water of the mice (male BALB/c mice, aged 8-10 weeks, n=6 per group) at a concentrations of 4% for a period of five days, followed by normal drinking water for seven days. Mice were given daily gastric gavage of the placebo (suspension medium Ora-Blend SF, Paddock Laboratories) or test compounds suspended in Ora-Blend SF using reusable feeding gavage needles (straight, 25 mm, 22G, 1.25 mm tip diameter, Fine Science Tools). Groups: 1) regular drinking water+placebo, 2) DSS+placebo, 3) DSS+sulfasalazine (111.5 mg/kg/day), 4) DSS+compound 13 (155.7 mg/kg/day). Mice were measured daily for changes in weight compared to the starting weight. Upon completion of the study, the colon was harvested from each mouse. Colons were weighed and measured for length. Sulfasalazine (2) was purchased from Sigma-Aldrich. Compound 13 was made as described in this paper. DSS was purchased from Affymetrix. In the second study, the percentage of DSS was reduced to 3% and the mice were exposed for 7 days, followed by 7 days with water.

### Example 18

### Cytokine Measurement

Mesenteric lymph nodes (MLNs) were harvested from the mice and gently disassociated using the frosted ends of sterilized glass slides to get a single cell suspension. The total cell suspension from the MLNs was then exposed to CD3/CD28 activation Dynabeads® purchased from Invitrogen using the manufacturer's protocol to stimulate proliferation of T cells and their associated cytokine production. Quantification of cytokines was measured with a multiplex panel kit from the Millipore Corporation using the manufacturer's protocol and LiquiChip (Luminex 100, Qiagen). The cytokines measured included IFN-γ, IL-17, TNF-α, IL-1β, IL-6 and MMP8 (the latter as a surrogate marker of mucosal neutrophil infiltration). Duplicates of samples were tested. MasterPlex QT software (Mirai-Bio) was used for data analysis.

The present disclosure has been described in connection with delivery to the colon of a drug or prodrug for treating IBD, microscopic colits, and eosinophilic colitis. However, the same technology may be employed for delivery of other drugs or prodrugs to the lower GI tract or other sites along the GI tract. The delivery system also may be used for colonic targeting of drugs currently existing on the market or in development for the treatment of other diseases and infections of the lower GI tract including, but not limited to, colorectal cancer (prevention and treatment), colonic polyps, acute and chronic diarrheal diseases, bacterial overgrowth, diverticulitis, irritable bowel syndrome and other types of functional abdominal pain disorders. The delivery system also may be used for delivering compounds isolated from probiotic strains directly to a targeted region in the lower GI tract.

A feature and advantage of the present disclosure is that the delivery system permits delivery of 5-ASA and other drugs and prodrugs in liquid form, which permits addition of the drugs or prodrugs to beverages, ice cream or the like. The lack of commercially available oral liquid formulations poses a frequent challenge in providing medications to pediatric patients, geriatric patients, patients with feeding tubes, and patients who cannot swallow solid dosage forms. Such a formulation would be invaluable for pediatric gastroenterologists for the treatment of early onset IBD in order eliminate difficulties related to the size of delayed-release tablets/capsules, increase palatability, compliance, and ultimately, clinical efficacy.

The foregoing discussion of the invention has been presented for purposes of illustration and description. The foregoing is not intended to limit the invention to the form or forms disclosed herein. Although the description of the invention has included description of one or more embodiments and certain variations and modifications, other variations and modifications are within the scope of the invention, e.g., as may be within the skill and knowledge of those in the art, after understanding the present disclosure. It is intended to obtain rights which include alternative embodiments to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter. All references cited herein are incorporated by reference in their entirety.

What is claimed is:

1. A composition comprising a disaccharide, wherein at least one hydroxyl group of said disaccharide is covalently linked to a linker, wherein a drug is covalently linked at a terminal portion of said linker with a functional group that is capable of releasing said drug in vivo.

2. The composition according to claim 1, wherein said disaccharide comprises a ketose monosaccharide, an aldose monosaccharide, or a combination thereof.

3. The composition according to claim 2, wherein said ketose monosaccharide comprises psicose, fructose, sorbose, tagatose, ribulose, or xylulose.

4. The composition according to claim 2, wherein said aldose monosaccharide comprises glucose, allose, altrose, mannose, gulose, idose, galactose, talose, ribose, arabinose, xylose, or lyxose.

5. The composition according to claim 2, wherein said disaccharide is selected from the group consisting of sucrose, lactose, allolactose, maltose, trehalose, cellobiose, lactulose, and chitobiose.

6. The composition according to claim 1 of the formula: 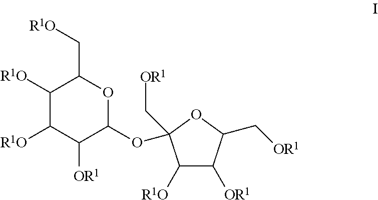
wherein each R¹ is independently hydrogen or said linker having chains of atoms selected from the group consisting of C, O and N, and said drug covalently linked at the terminal portion of said linker with said functional group that is capable of releasing said drug in vivo, provided at least one R¹ is said linker.

7. The composition according to claim 6, wherein said linker comprises at least one cyclic moiety, wherein each of said cyclic moiety is independently selected from the group consisting of aryl, heteroaryl, cycloalkyl, heterocycloalkyl, and a combination thereof, each of which is optionally substituted.

8. The composition according to claim 6, wherein said linker comprises a moiety of the formula: 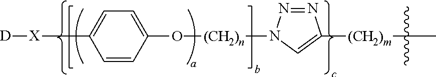
wherein
each of a, b, and c is independently 0 or 1;
each of m and n is an integer from 1 to 15, provided that the total number of atoms in said linker is no more than 30;
D is a drug; and
X is said functional group that is capable of releasing said drug in vivo.

9. The composition according to claim 8, wherein a, b, and c is 1, m is 4 and n is 8.

10. The composition according to claim 1 of the formula: 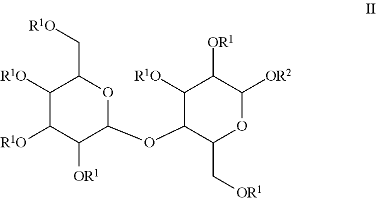
wherein R² is H, a hydroxyl protecting group or R¹, and each R¹ is independently hydrogen or said linker having chains of atoms selected from the group consisting of C, O and N, and said drug covalently linked at the terminal portion of said linker with said functional group that is capable of releasing said drug in vivo, provided at least one R¹ is said linker.

11. The composition according to claim 10, wherein said linker comprises at least one cyclic moiety, wherein each of said cyclic moiety is independently selected from the group consisting of aryl, heteroaryl, cycloalkyl, heterocycloalkyl, and a combination thereof, each of which is optionally substituted.

12. The composition according to claim 10, wherein said linker comprises a moiety of the formula: 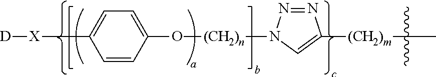
wherein
each of a, b, and c is independently 0 or 1;
each of m and n is an integer from 1 to 15, provided that the total number of atoms in said linker is no more than 30;
D is a drug; and
X is said functional group that is capable of releasing said drug in vivo.

13. The composition according to claim 12, wherein a, b, and c is 1, m is 4 and n is 8.

14. The composition according to claim 1 of the formula: 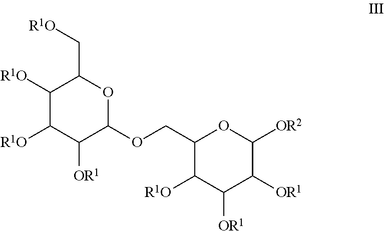
wherein R² is H, a hydroxyl protecting group or R¹, and each R¹ is independently hydrogen or said linker having chains of atoms selected from the group consisting of C, O and N, and said drug covalently linked at the terminal portion of said linker with said functional group that is capable of releasing said drug in vivo, provided at least one R¹ is said linker.

15. The composition according to claim 14, wherein said linker comprises at least one cyclic moiety, wherein each of said cyclic moiety is independently selected from the group consisting of aryl, heteroaryl, cycloalkyl, heterocycloalkyl, and a combination thereof, each of which is optionally substituted.

16. The composition according to claim 15, wherein said linker comprises a moiety of the formula: 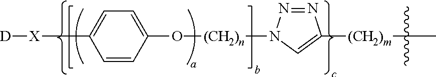
wherein
each of a, b, and c is independently 0 or 1;
each of m and n is an integer from 1 to 15, provided that the total number of atoms in said linker is no more than 30;
D is a drug; and
X is said functional group that is capable of releasing said drug in vivo.

17. The composition according to claim 16, wherein a, b, and c is 1, m is 4 and n is 8.

18. The composition according to claim 1 of the formula: 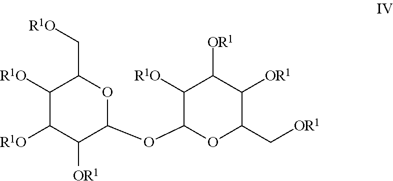
wherein each R¹ is independently hydrogen or said linker having chains of atoms selected from the group consisting of C, O and N, and said drug covalently linked at the terminal portion of said linker with said functional group that is capable of releasing said drug in vivo, provided at least one R¹ is said linker.

19. The composition according to claim 14, wherein said linker comprises at least one cyclic moiety, wherein each of said cyclic moiety is independently selected from the group consisting of aryl, heteroaryl, cycloalkyl, heterocycloalkyl, and a combination thereof, each of which is optionally substituted.

20. The composition according to claim 15, wherein said linker comprises a moiety of the formula: 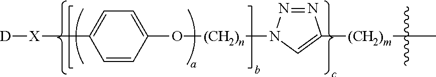
wherein
each of a, b, and c is independently 0 or 1;
each of m and n is an integer from 1 to 15, provided that the total number of atoms in said linker is no more than 30;
D is a drug; and
X is said functional group that is capable of releasing said drug in vivo.
